(19) United States
(12) Patent Application Publication
(10) Pub. No.: US 2026/0317403 A1
(43) Pub. Date: Oct. 15, 2026

(54) SARS-COV2 NEUTRALIZING SINGLE DOMAIN ANTIBODY CONSTRUCTS

(71) Applicant: THE REGENTS OF THE UNIVERSITY OF CALIFORNIA, Oakland, CA (US)

(72) Inventors: Aashish Manglik, San Francisco, CA (US); Yue Wan, San Francisco, CA (US)

(21) Appl. No.: 19/104,622

(22) PCT Filed: Aug. 17, 2023
(86) PCT No.: PCT/US2023/072414

§ 371 Date: Feb. 18, 2025

Related U.S. Application Data
(60) Provisional application No. 63/399,617, filed on Aug. 19, 2022.

(51) Int. Cl. C07K 16/104; A61K 9/19

(52) U.S. Cl. CPC C07K 16/104; A61K 9/19; C07K 2317/569

(57) ABSTRACT

Antibodies, including single-domain antibodies, that bind to SARS-CoV2 virus and methods of treatment using single-domain antibodies that bind to SARS-CoV2 virus are provided.

### CROSS REFERENCE TO RELATED APPLICATIONS

This application claims the benefit of U.S. Provisional Application No. 63/399,617, filed Aug. 19, 2022, which is incorporated herein by reference in its entirety.

### BACKGROUND OF THE INVENTION

Severe acute respiratory syndrome coronavirus 2 or “SARS-CoV-2” is a virus strain that causes coronavirus disease 2019 (COVID-19). See, e.g., Gorbalenya A E, et al. Nature Microbiology. 5 (4): 536-544 (March 2020). Therapeutic treatments to address the global pandemic are needed.

### BRIEF DESCRIPTION OF THE DRAWINGS

FIGS. 1A and 1B collectively depict the general strategy for blocking the entry of the SARS-CoV2 virus (“SC2 virus”). As generally discussed herein, the spike protein of the SC2 virus forms a trimeric structure that binds to the extracellular domain of the ACE2 receptor on human cells at a location deemed the spike receptor binding domain (RBD). FIG. 1A depicts a space filling model and FIG. 1B uses a ribbon diagram. FIG. 1B shows that by blocking the ACE2—spike protein interaction, the SC2 virus can no longer enter the host cells.

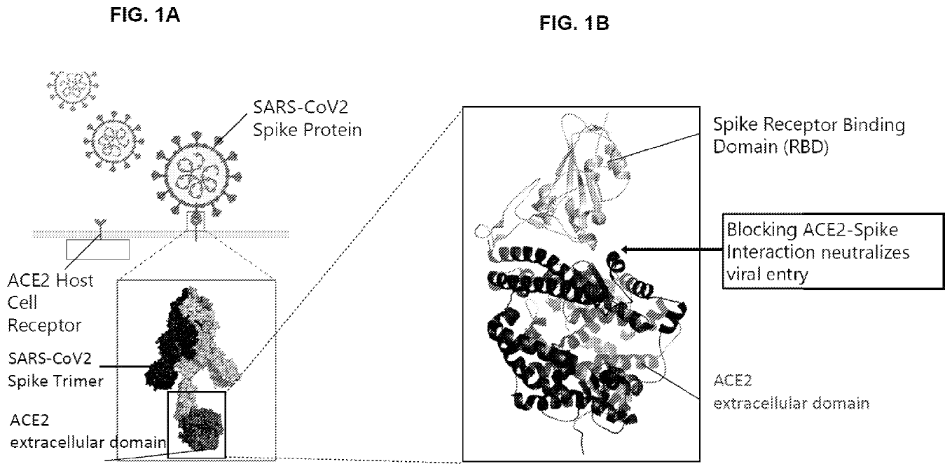

FIG. 2 depicts amino acid sequences of full length MASC proteins AeroNab6 and hmONb2.

FIG. 3 depicts amino acid sequences of full length hmONab2, vhCDRs 1-3, and framework regions 1-4.

FIGS. 4A and 4B collectively show the surface plasmon resonance results of MASC protein binding to Omicron BA.1.1.529 Spike protein.

### DETAILED DESCRIPTION OF THE INVENTION

### A. Definitions

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to the Spike trimeric protein as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antigen binding domain (SBD) having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹³ M, at least about 10⁻¹⁴ M, at least about 10⁻¹⁵ M or greater, where KD refers to a dissociation rate of a particular ABD-antigen interaction. Typically, an ABD that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including the renumerated anti-Spike antigen binding domains of the invention) as used herein is meant a polypeptide that is subsequently modified to generate a variant. In this case, for example, any one of the starting clones can be considered a “parent polypeptide”. Parent polypeptide may refer to the polypeptide itself, compositions that comprise the parent polypeptide, or the amino acid sequence that encodes it.

The polypeptides of the invention have at least about 90%, 91, 92, 92, 94, 95, 96, 97, 98, 99, 99.2. 99.4. 99.6. 99.8 or 100% sequence identity with a sequence set forth herein.

By “position” as used herein is meant a location in the sequence of a protein. Positions may be numbered sequentially, or according to an established format.

By “variable heavy domain” or “VH domain” or “VHH domain” herein is meant the region of the antigen binding domain that contains the CDRs. The molecules discussed herein do not contain VL domains. In these embodiments, each VH is composed of three hypervariable regions (“complementary determining regions,” “CDRs”) and four “framework regions”, or “FRs”, arranged from amino-terminus to carboxy-terminus in the following order: FR1-vhhCDR1-FR2-vhhCDR2-FR3-vhhCDR3-FR4. The vhFR regions self-assemble to form the sdABD that are Fv domains. By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). sdABDs are distinguished from single domain antibodies by the lack of the constant domains (in the case of camelid antibodies, the CH2-CH3 domains).

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the IMGT numbering system is generally used when referring to a residue in the variable domain.

The present invention provides a large number of different CDR sets which can be assembled into sdABDs. In the context of a single domain ABD (“sdABD”), a CDR set is only three CDRs; these are sometimes referred to in the art as “VHH” domains as well.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

### B. Introduction

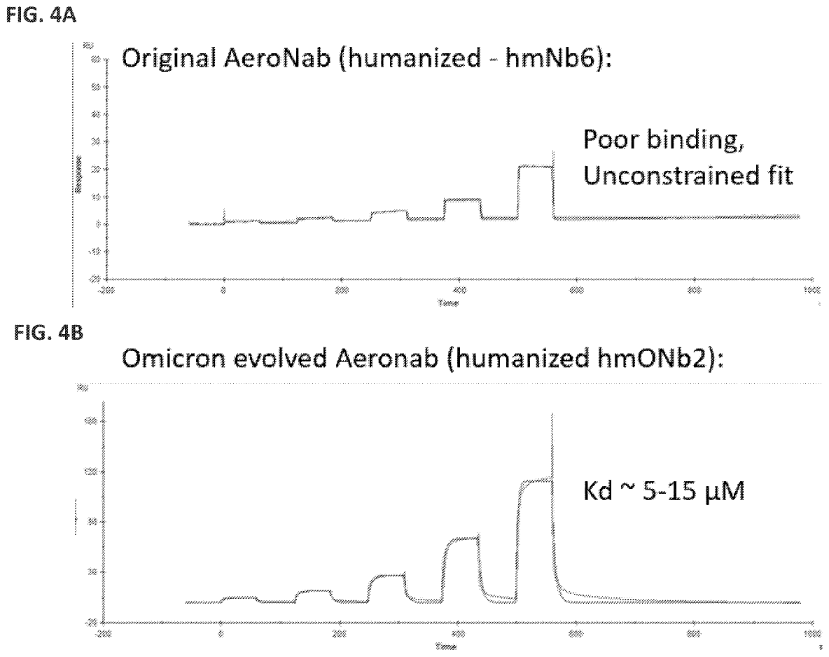

As is known in the art, coronaviruses are enveloped positive-stranded RNA viruses that relies on membrane fusion as an early step for entering host cells. Additionally, the surfaces of many coronaviruses, and SARS-CoV2 Omicron BA.1.1.529 in particular, are decorated with a Spike glycoprotein. The Spike protein forms a homotrimeric complex of three identical Spike protein monomers that can be functionally categorized as having two distinct subunits, S1 and S2. The S1 subunit contains the receptor binding domain (RBD) which binds to the ACE2 receptor on human cells, while the S2 subunit is involved in the fusion of the viral and cellular membranes. The RBDs of the trimeric complex can be in two different conformations: an extended, or “up” conformation (sometimes also referred to herein as the “on” conformation), as depicted in FIG. 4, that is accessible for binding to the ACE2 complex, and a “down” or “off” conformation, again depicted in FIG. 4, which represents a receptor-inaccessible state. That is, the Spike protein cannot bind to the ACE2 receptor and infect cells when in the “down” conformation.

The present invention is directed to antigen binding domains that not only bind to the Spike protein, but bind in such a way as to “lock” the Spike protein in the “off” or “down” position with extremely high affinity. The binding of the MASC proteins of the invention actually bind to two different RBDs (of the three present in the trimer), thus not only keeping the Spike protein “off” but also occupying a part of the ACE2 binding site to further prevent membrane fusion and infection.

The present invention provides Multivalent Anti-SARS-CoV2 (“MASC”) fusion proteins to antigen binding domains (ABDs) that bind in a multivalent way to the trimeric Spike protein of the SARS-CoV2 virus, Omicron BA.1.1.529 Spike protein, with very high affinity. The antigen binding domains are based on single domain antibodies (sdAbs) that contain a single variable heavy domain (frequently referred to in the field as “VHH” domains) instead of the typical variable heavy and variable light domains of traditional antibodies. The single domain antigen binding domains (sdABDs) confer a number of advantages in their use to bind to viral proteins, as they are significantly smaller than traditional ABDs, have generally high thermal stability, and increased solubility, as further discussed below. Exemplary disclosures of sdAbs and their use as prophylactic or therapeutic for SARS-CoV2 diseases are provided in International Patent Publication WO/2021/231651, which is incorporated herein by reference in its entirety.

Additionally, as has been shown for other sdABDs, the sdABDs of the present invention can be assembled into multimeric structures such as dimers and trimers, similar to other “Nanobodies™”; see generally U.S. Pat. No. 9,834,595, and International Patent Publication WO/2021/231651, both of which are incorporated herein by reference in their entireties. Thus, the present invention provides multivalent anti-SARS-CoV2 (“MASC”) fusion proteins that contain sdABDs linked together through domain linkers as is more fully described below that bind the Spike protein and prevent viral entry into human cells via the ACE2 receptor, as is further discussed below.

In these multivalent embodiments, the fusion proteins of the invention have a several different components, generally referred to herein as domains, which are linked together in a variety of ways depending on the format. Some of the domains are binding domains, that each bind to the target Spike protein, and some are domain linkers. Thus, the present invention provides for MASC proteins that comprise a single sdABD that binds the RBD, as well as MASC fusion proteins that contain two sdABDs linked using a domain linker (sdABD-domain linker-sdABD) and MASC fusion proteins that contain three sdABDs linked with domain linkers (sdABD-domain linker-sdABD-domain linker-sdABD). As discussed below, these domain linkers can be the same or different.

Another distinct advantage of the MASC fusion proteins of the invention is that due to their significant thermal and structure stability, the MASC fusion proteins can be lyophilized and/or aerosolized while retaining binding and neutralization functions. The possibility of administering these MASC fusion proteins directly to the pulmonary system is particularly useful in the case of the SARS-CoV2 virus, as it is known to act specifically in the lungs.

In some embodiments, the MASC proteins of the invention comprise CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more amino acid substitutions; (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Additionally included in the definition of MASC proteins are proteins that share identity to the MASC proteins enumerated herein. That is, in certain embodiments, an MASC protein according to the invention comprises heavy variable region comprising amino acid sequences that are homologous to amino acid sequences of preferred MASC proteins, respectively, wherein the proteins retain the desired functional properties of the reference MASC proteins. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the protein molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In general, the percentage identity for comparison between MASC proteins is at least 75%, at least 80%, at least 90%, with at least about 95, 96, 97, 98 or 99% percent identity being preferred. The percentage identity may be along the whole amino acid sequence, for example the entire heavy or light chain or along a portion of the chains. For example, included within the definition of the MASC proteins of the invention are those that share identity along the entire variable region (for example, where the identity is 95 or 98% identical along the variable regions), or along the entire constant region, or along just the Fc domain.

In addition, also included are sequences that may have the identical CDRs but changes in the variable domain (or entire heavy or light chain). For example, MASC proteins include those with CDRs identical to those shown in hmONb2 but whose identity along the variable region can be lower, for example 95 or 98% percent identical.

Accordingly, the present invention provides MASC proteins and MASC fusion proteins as is further described herein.

### C. Multivalent Anti-SARS-CoV2 (“MASC”) Proteins

Accordingly, the present invention provides MASC proteins that can take several different formats. As discussed herein, the MASC proteins can be a single sdABD as outlined herein, sometimes referred to herein as “monomeric MASC proteins”. MASC proteins can also be linked together to form dimers and trimers as discussed herein. In these embodiments, the dimers and trimers are referred to generally as “MASC fusion proteins”, and there are domain linkers between the monomers.

As will be appreciated by those in the art and more fully described below, in addition to multimers of a single sdABD, multimers can also be made using sdABDs with different binding affinities or properties. That is, a dimeric MASC fusion protein can be “homodimeric”, with the sdABDs having the identical CDRs and/or sequence, or “heterodimeric”, where one sdABD has one set of CDRs and the other has a different set of CDRs. Similarly, trimeric MASC fusion proteins can be homotrimers, or they can be heterotrimers, either utilizing two different sdABDs with different CDRs (two of one and one of the other in the trimer), or heterotrimers with three different CDR sets.

Additionally, as more fully discussed below, the MASC fusion proteins can also include an additional domain that serves to extend the half-life of the MASC protein in plasma. Thus, for example, monomeric, dimeric or trimeric MASC proteins can be fused to a half-life extension domain.

As discussed herein, 1 MASC protein clone was originally made. The clone bound the spike trimer of Omicron BA.1.1.529.

Furthermore, as discussed herein, any of the parental MASC proteins can undergo affinity maturation. An exemplary example is the affinity maturation of AeroNab6, discussed herein. An affinity maturation campaign resulted in a number of changes in the vhhCDRs, all of which can be combined.

Similarly, as will be appreciated by those in the art, the parental MASC proteins or affinity matured MASC proteins can also be humanized. Humanization techniques are well known in the art.

In some embodiments, the humanized MASC proteins include but are not limited to hmONb2 that binds to SARS-CoV2 variant Omicron BA.1.1.529.

Additionally, it is known in the art that there can be immunogenicity in humans originating from the C-terminal sequences of certain ABDs. Accordingly, in general, when the C-terminus of the constructs terminates in an sdABD such as depicted here, a histidine tag (either His6 or His10) can be used. These in some cases can be used as purification tags as well, but these sequences can also be used to reduce immunogenicity in humans, as is shown by Holland et al., DOI 10.1007/s10875-013-9915-0 and WO2013/024059, both of which are incorporated herein by reference in their entireties.

### 1. Monomeric Constructs

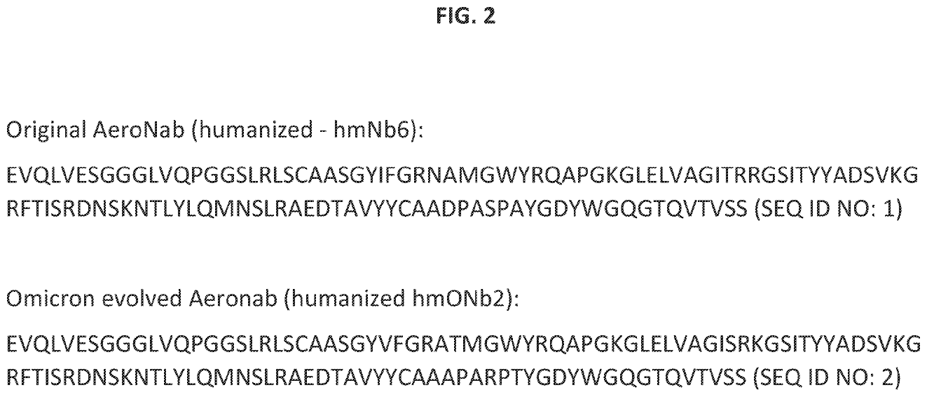

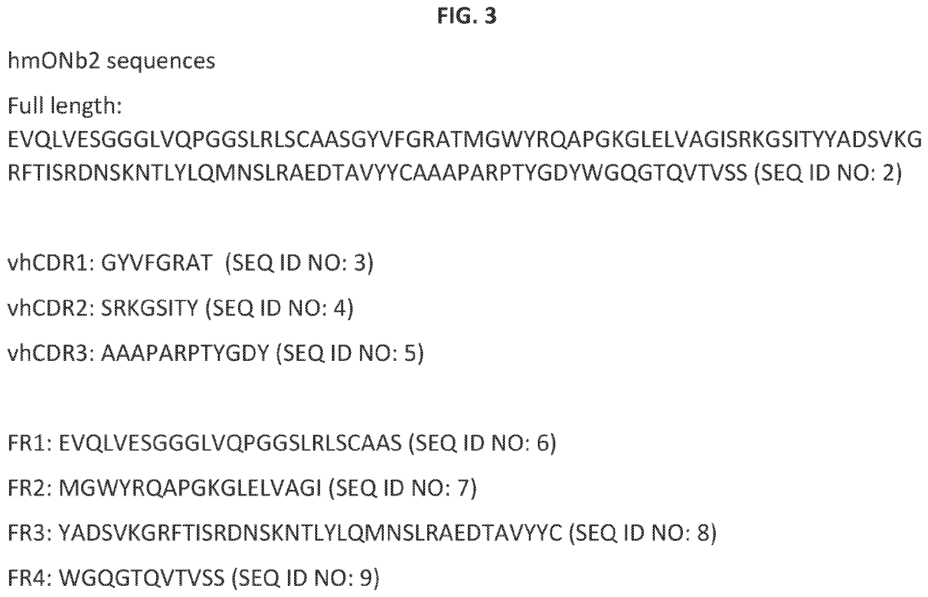

In some embodiments, the MASC protein is a single sdABD, as generally depicted in FIGS. 2 and 3, and thus is a composition comprising a sdABD comprising, from N- to C-terminal, FR1-vhCDR1-FR2-vhCDR2-FR3-vhCDR3-FR.

In some embodiments, the sdABD has a set of a three CDRs where vhCDR1 has SEQ ID NO:3, vhCDR2 has SEQ ID NO:4, and vhCDR3 has SEQ ID NO:5.

In this embodiment, the framework regions can have SEQ ID NO:6 (FR1), SEQ ID NO:7 (FR2), SEQ ID NO:8 (FR3) and SEQ ID NO:9 (FR4), or can be different.

In a particularly useful embodiment, the MASC protein is “hmONb2” and has the sequence set forth as follows: EVQLVESGGGLVQPGGSLRLSCAASGYVFGRATMGWYRQAPGKGLELVAGISRKGSITYY ADSVKGRFTISRDNSKNTLYLQMNSLRAEDTAVYYCAAAPARPTYGDYWGQGTQVTVSS (SEQ ID NO:2).

### 2. Dimeric Constructs

In some embodiments, the MASC protein is a MASC fusion protein and contains two sdABDs, as generally depicted in FIGS. 2 and 3, and thus is a composition comprising a sdABD comprising, from N- to C-terminal, FR1-vhhCDR1-FR2-vhhCDR2-FR3-vhhCDR3-FR4-domain linker-FR1-vhhCDR1-FR2-vhhCDR2-FR3-vhhCDR3-FR4.

In many embodiments, the two sdABDs that make up the dimer are the same, and the domain linker is selected from (GGGGS)₃ and (GGGGS)₄. In some embodiments, the MASC comprises two hmONb2.

In some dimeric embodiments, the two sdABDs each have a set of three CDRs where vhCDR1 has SEQ ID NO:3, vhCDR2 has SEQ ID NO:4, and vhCDR3 has SEQ ID NO:5. In this embodiment, the framework regions can have SEQ ID NO:6 (FR1), SEQ ID NO:7 (FR2), SEQ ID NO:8 (FR3) and SEQ ID NO:9 (FR4), and the domain linker is selected from (GGGGS)₃ and (GGGGS)₄.

### 3. Trimeric Constructs

In some embodiments, the MASC protein is a MASC fusion protein and contains three sdABDs, as generally depicted in FIGS. 2 and 3, and thus is a composition comprising a sdABD comprising, from N- to C-terminal, FR1-vhhCDR1-FR2-vhhCDR2-FR3-vhhCDR3-FR4-domain linker-FR1-vhhCDR1-FR2-vhhCDR2-FR3-vhhCDR3-FR4.

In many embodiments, the three sdABDs that make up the trimer are the same, and the domain linker is selected from (GGGGS)₃ and (GGGGS)₄. In some embodiments, the MASC comprises three hmONb2.

In some trimeric embodiments, the two sdABDs each have a set of three CDRs where vhCDR1 has SEQ ID NO:3, vhCDR2 has SEQ ID NO:4, and vhCDR3 has SEQ ID NO:5. In this embodiment, the framework regions can have SEQ ID NO:6 (FR1), SEQ ID NO:7 (FR2), SEQ ID NO:8 (FR3) and SEQ ID NO:9 (FR4), and the domain linker is selected from (GGGGS)₃ and (GGGGS)₄.

### 4. Domain Linkers

In embodiments utilizing multimeric MASC proteins, the monomers are linked recombinantly using “domain linkers”. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. For example, the distance between the N- and C-termini of individual AeroNab6 monomers bound to spike ECD in the “down” state is 51 Å. This requires ≥15 amino acids to bridge individual subunits to simultaneously engage multiple RBD monomers. Thus, in some embodiments, the linker are (GGGGS)₃ and (GGGGS)₄. Both (GGGGS)₃ and (GGGGS)₄ linkers in the trimeric constructs show both good binding to the spike protein trimer as measured by SPR.

### 5. Half-Life Extension Domains

The MASC proteins optionally include half-life extension domains, that allow for increased half-life in physiological environments such as plasma and lung tissue. Such domains are contemplated to include, but are not limited to, HSA binding domains, either scFvs or sdABDs, as well as all or part of human serum albumin, as discussed below.

Human serum albumin (HSA) (molecular mass ~67 kDa) is the most abundant protein in plasma, present at about 50 mg/ml (600 uM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise a half-life extension domain, for example a domain which specifically binds to HSA, that is attached either N- or C-terminal to the MASC protein. In many embodiments, the half-life extension domain is a single domain antigen binding domain from a single domain antibody that binds to HSA. This domain is generally referred to herein as “sdABD” to human HSA (sdABD-HSA), or alternatively “sdABD(½)”, to distinguish these binding domains from the sdABDs to the spike protein. Suitable sdABD-HSA domains are well known in the art, see for example U.S. Pat. No. 8,703,131, the sequences of all sdABD-HSA domains therein (“ALB”, including specifically ALB1, ALB3, ALB4, ALB5, ALB6, ALB7, ALB8, ALB9 and ALB10) are expressly incorporated by reference. Similarly, U.S. Pat. No. 10,100,106 and WO/2021/231651 contain additional single domain albumin binding domains, the sequences of which are also specifically incorporated by reference herein, including SEQ ID NOs:4, 7, 9, 26 and 27.

Another suitable half-life domain that can be fused to the MASC proteins is all or part of human HSA itself, again, either N- or C-terminal. HSA is a relatively small protein, roughly 65 amino acids long, and can be fused to one or more of the monomeric MASC proteins as will be appreciated by those in the art.

The half-life extension domain of an antigen binding protein provides for altered pharmacodynamics and pharmacokinetics of the MASC proteins. As above, the half-life extension domain extends the elimination half-time. The half-life extension domain also alters pharmacodynamic properties including alteration of tissue distribution, penetration, and diffusion of the antigen-binding protein.

### D. Method of Making MASC Proteins

The MASC proteins and fusion proteins of the invention are made as will generally be appreciated by those in the art and outlined below. Exemplary methods of making MASC proteins are provided in WO/2021/231651, which is incorporated herein by reference in its entirety.

The invention provides nucleic acid compositions that encode the MASC compositions of the invention. As is known in the art, the nucleic acids encoding the compositions of the invention can be incorporated into expression vectors as is known in the art, and depending on the host cells used to produce the MASC proteins of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells, 293 cells), finding use in many embodiments.

The MASC proteins, including MASC fusion proteins, of the invention are made by culturing host cells comprising the expression vector(s) as is well known in the art under conditions that result in the expression of the proteins, followed by purification.

### E. Formulations

Formulations of the MASC proteins used in accordance with the present invention are prepared for storage by mixing the proteins having the desired degree of purity with optional pharmaceutically acceptable carriers, excipients or stabilizers (as generally outlined in Remington's Pharmaceutical Sciences 16th edition, Osol, A. Ed. [1980]), in the form of lyophilized formulations or aqueous solutions.

### F. Administration of the MASC Proteins

The compositions of the invention comprising MASC proteins and MASC fusion proteins that are administered to a patient to prevent, treat or neutralize the SC2 virus or SC2 viral infection, particularly Omicron variant infection such as the one caused by BA.1.1.529 strain, in a patient. As recently reported, it appears that the highest ACE2 expression in humans is in the nose with decreasing expression throughout the lower respiratory tract, corresponding to a gradient of SC2 infection from high in the proximal nasal passages to lower in the distal areas such as peripheral lung (see Hou et al, https://doi.org/10.1016/j.cell.2020.05.042), supporting an early hypothesis that nasal surfaces might be the dominant initial site for infection. The infection then proceeds to the lungs.

Accordingly, as will be appreciated by the art, there are a number of different administration routes which can be utilized to administer the MASC proteins of the invention, including, but not limited to, pulmonary delivery using inhalation techniques, intranasal delivery using particular formulations and intravenous administration, as outlined herein.

### 1. Inhalation Therapy

Thus, in some embodiments, the MASC proteins are administered to a patient's pulmonary system, including the lungs. Thus, the invention provides for the delivery of the MASC proteins (including MASC fusion proteins) of the invention to the respiratory tract.

One benefit of the MASC proteins of the invention is that they are extremely stable and thus can be lyophilized, as is known in the art. The lyophilized proteins can then be reconstituted at a later date into a liquid formulation and then aerosolized through nebulization for direct delivery to the patient's pulmonary system. See for example U.S. Pat. No. 9,393,304 which describes a number of lyophilization techniques, conditions and formulations for Nanobodies™ that are for inhalation therapy.

In certain embodiments, the formulations can be administered using nebulizers. Examples of nebulizers include, in non-limiting examples, jet nebulizers, ultrasonic nebulizers, and vibrating mesh nebulizers. These classes use different methods to create an aerosol from a liquid. In general, any aerosol-generating device that can maintain the integrity of the protein in these formulations is suitable for delivery of formulations as described herein.

In some embodiments, a vibrating-mesh nebulizers is used. Vibrating-mesh nebulizers are divided into passively and actively vibrating-mesh devices (Newman 2005, J. Appl. Ther. Res. 5: 29-33). Passively vibrating-mesh devices (e.g. Omron MICROAIR® NE-U22 nebulizer) employ a perforated plate having up to 6000 micron sized holes. A vibrating piezo-electric crystal attached to a transducer horn induces “passive” vibrations in the perforated plate positioned in front of it, resulting in extrusion of fluid through the holes and generation of the aerosol. Actively vibrating-mesh devices (e.g. AERONEB® Pro nebulizer) may employ a “micropump” system which comprises an aerosol generator consisting of a plate with up to 1000 dome-shaped apertures and a vibrating element which contracts and expands on application of an electric current. This results in upward and downward movements of the mesh by a few micrometers, extruding the fluid and generating the aerosol. Other examples of vibrating-mesh nebulizers include the Akita2 Apixneb (Activaero, now Vectura, Germany), EFLOW® (PARI GmbH, Grafelingen, Germany; see also U.S. Pat. No. 5,586,550), AERONEB® (Aerogen, Inc., Sunnyvale, Calif.; see also U.S. Pat. Nos. 5,586,550; 5,938,117; 6,014,970; 6,085,740; 6,205,999), or the FOX nebulizer (Activaero, now Vectura, Germany), all adapted for pediatric use.

In some embodiments, a continuous flow nebuliser is used, particularly in cases where COVID19 patients may require oxygen as well, so continuous flow can be used to maintain a continuous oxygen or air supply to the patient. Accordingly, the nebulizer can be used with or without additional air or O₂ flow. Preferably, the nebulizer is used with additional air or O₂ flow, such as a flow of 2 L/min additional air or O₂.

An exemplary inhalation device for delivering the polypeptide of the invention to a patient may comprises (a) an aerosol generator with a vibratable mesh; (b) a reservoir for a liquid to be nebulised, said reservoir being in fluid connection with the vibratable mesh; (c) a gas inlet opening; (d) a face mask, having a casing, an aerosol inlet opening, a patient contacting surface, and a one-way exhalation valve or a two-way inhalation/exhalation valve in the casing having an exhalation resistance selected in the range from 0.5 to 5 mbar; and (e) a flow channel extending from the gas inlet opening to the aerosol inlet opening of the face mask, the flow channel having a lateral opening through which the aerosol generator is at least partially inserted into the flow channel, and a constant flow resistance between the gas inlet opening and the aerosol inlet opening of the face mask at a flow rate of 1 to 20 L/min.

Additional methods for delivery to the respiratory tract and/or delivery by inhalation are known to the skilled person and are e.g. described in the handbook “Drug Delivery: Principles and Applications” (2005) by Binghe Wang, Teruna Siahaan and Richard Soltero (Eds. Wiley Interscience (John Wiley & Sons)); in “Pharmacology PreTest™ (11.sup.th Ed.) Self-Assessment and Review” by Rosenfeld G. C., Loose-Mitchell D. S.; and in “Pharmacology” (3.sup.rd Edition) by Lippincott Williams & Wilkins, New York; Shlafer M. McGraw-Hill Medical Publishing Division, New York; Yang K. Y., Graff L. R., Caughey A. B. Blueprints Pharmacology, Blackwell Publishing.

The present invention also relates to a pharmaceutical device suitable for the delivery by inhalation of the MASC proteins of the invention and suitable in the use of a composition comprising the same. The present invention, accordingly, relates to such a device comprising the MASC proteins of the invention at the selected dose.

Various inhalation systems are e.g. described on pages 129 to 148 in the review (“Pulmonary Drug Delivery”, Bechtold-Peters and Luessen, eds., supra). In the method of the present invention, the device is an inhaler for liquids (e.g. a suspension of fine solid particles or droplets) comprising the polypeptide of the invention. Preferably this device is an aerosol delivery system or a nebulizer comprising the polypeptide of the invention.

The aerosol delivery system used in the method of the invention may comprise a container comprising the composition of the invention and an aerosol generator connected to it. The aerosol generator is constructed and arranged to generate an aerosol of the composition of the invention.

### 2. Intranasal Administration

As discussed above, it appears that the nasal cavity and nasal surfaces might be the dominant initial site for SC2 viral infection, as evidenced by the high ACE2 expression patterns.

Accordingly, in some embodiments the MASC proteins, including MASC fusion proteins, are administered via nasal administration as a nasal spray. There are a wide variety of delivery systems for intranasal administration of the MASC proteins, ranging from simple drops or sprays to unit dosing systems for liquids; see for example Marx et al., Intranasal Drug Administration—An Attractive Delivery Route for Some Drugs; DOI: 10.5772/59468. As above, the MASC proteins can be lyophilized and then reconstituted for nasal administration or administered directly as a liquid with lyophilization.

### 3. Intravenous Administration

Additionally, as will be appreciated by those in the art, the MASC proteins of the invention can also be administered intravenously.

### G. Methods of Diagnosing SC Viral Infection

In some embodiments, one or more MASC proteins as described herein can be used to detect SARS-CoV2 in a biological or non-biological sample. For example, MASC proteins reagents can be used in assays to detect the presence or absence of, or protein expression levels, for SARS-CoV2 using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the protein concentration, are suitable for use in the methods of the present invention. Nephelometry assays are commercially available from Beckman Coulter (Brea, CA; Kit #449430) and can be performed using a Behring Nephelometer Analyzer (Fink et al., J. Clin. Chem. Clin. Biochem., 27:261-276 (1989)).

Specific immunological binding of the MASC proteins to SARS-CoV2 can be detected directly or indirectly. Direct labels include fluorescent or luminescent tags, metals, dyes, radionuclides, and the like, attached to the antibody. A MASC protein labeled with iodine-125 (125I) can be used. A chemiluminescence assay using a chemiluminescent antibody specific for the nucleic acid is suitable for sensitive, non-radioactive detection of protein levels. A MASC proteins labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a (3-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, MO).

A signal from the direct or indirect label can be analyzed, for example, using a spectrophotometer to detect color from a chromogenic substrate; a radiation counter to detect radiation such as a gamma counter for detection of 125I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, CA) in accordance with the manufacturer's instructions. If desired, the assays of the present invention can be automated or performed robotically, and the signal from multiple samples can be detected simultaneously.

The MASC proteins can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

### H. Methods of Screening for Antigen Binding Domains

Also provided herein are methods to screen for other ABDs that compete for binding with the present MASC proteins. The term “compete”, as used herein with regard to an ABD, means that a first ABD, or an antigen-binding portion thereof, competes for binding with a second ABD, or an antigen-binding portion thereof, where binding of the first ABD with its cognate epitope is detectably decreased in the presence of the second ABD compared to the binding of the first ABD in the absence of the ABD antibody. The alternative, where the binding of the second ABD to its epitope is also detectably decreased in the presence of the first ABD, can, but need not be the case. That is, a first ABD can inhibit the binding of a second ABD to its epitope without that second ABD inhibiting the binding of the first ABD to its respective epitope. However, where each ABD detectably inhibits the binding of the other ABD with its cognate epitope or ligand, whether to the same, greater, or lesser extent, the ABDs are said to “cross-compete” with each other for binding of their respective epitope(s). Both competing and cross-competing ABD are encompassed by the present invention. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing ABDs are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using 1-125 label (see Morel et al., Molec. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

Competitive binding assays can be used to identify antibodies that compete with an antibody described herein for specific binding to the SARS-CoV2 virus. Any of a number of competitive binding assays known in the art can be used to measure competition between two antibodies to the same antigen. Briefly, the ability of different antibodies to inhibit the binding of another antibody is tested. For example, antibodies can be differentiated by the epitope to which they bind using a sandwich ELISA assay. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### II. EXAMPLES

### Example 1: Identification and Characterization of Parental MASC Protein

### (a) Binding of Yeast-Surface Displayed Nanobodies to Omicron BA.1.1.529 Spike Protein

Yeast displaying a single nanobody clone on the surface were assessed for binding to purified fluorescently labeled Omicron BA.1.1.529 Spike protein by flow cytometry. Yeast were incubated with Spike ectodomain (Spike ECD) or the receptor binding domain (RBD) labeled with Alexa647 for 30 minutes at 25 C. After extensive washing of yeast by repeated centrifugation and resuspension, the amount of SARS-CoV2 Spike ectodomain bound to yeast cells was measured by flow cytometry. Nanobody clones that bind to Omicron BA.1.1.529 Spike protein showed strong fluorescence signal in the Alexa647 channel. Binding to SARS-CoV2 Spike ectodomain was decreased in the presence of purified ACE2-Fc, indicative of an epitope that is competitive with human ACE2. The nanobody is further humanized and the humanized sequence (hmONb2) is provided in FIGS. 2 and 3.

### (b) Surface Plasmon Resonance of Nanobodies to Spike Ectodomain (ECD).

Stabilized SARS-CoV2 ectodomain bearing a C-terminal 8× histidine tag and a Twin-strep-tag was expressed in Expi293 cells and purified using metal-affinity chromatography and size exclusion chromatography. This antigen was captured on a Cytiva surface plasmon resonance chip via Streptactin XT to analyze the kinetic properties of nanobodies hmONb2 and humanized AeroNab6. The results are shown in FIG. 4.

What is claimed is:

1. A composition comprising a single domain antigen binding domain (sdABD) that binds to Omicron BA.1.1.529 Spike protein, wherein said sdABD comprises, from N- to C-terminal, FR1-vhCDR1-FR2-vhCDR2-FR3-vhCDR3-FR, wherein said vhCDR1 comprises an amino acid sequence of SEQ ID NO:3, said vhCDR2 comprises an amino acid sequence of SEQ ID NO:4, and said vhCDR3 comprises an amino acid sequence of SEQ ID NO:5.

2. A composition according to claim 1, wherein said FR1 comprises an amino acid sequence of SEQ ID NO:6, said FR2 comprises an amino acid sequence of SEQ ID NO:7, said FR3 comprises an amino acid sequence of SEQ ID NO:8, and said FR4 comprises an amino acid sequence of SEQ ID NO:9.

3. A composition according to claim 1 or 2, wherein said sdABD comprises an amino acid sequence of SEQ ID NO:2.

4. A composition according to any of claims 1 to 3, wherein said composition further comprises a half-life extension domain covalently attached to said sdABD using a domain linker.

5. A composition according to claim 4, wherein said half-life extension domain is selected from the group consisting of an anti-human serum albumin (HSA) sdABD and all or part of HSA.

6. A nucleic acid encoding the composition of any of claims 1 to 5

7. An expression vector comprising the nucleic acid of claim 6.

8. A host cell comprising the expression vector of claim 7.

9. A method of making a composition according to any of claims 1 to 5 comprising culturing the host cell of claim 8 under conditions whereby said composition is expressed and recovering said composition.

10. A method of treating or preventing an infection by SARS-CoV2 virus comprising administering to a human the composition of any of claims 1 to 5, thereby treating or preventing said infection.

11. A method of neutralizing the SARS-CoV2 virus comprising administering to a human the composition of any of claims 1 to 5, thereby neutralizing said virus.

12. A method according to claim 10 or 11, wherein said SARS-CoV2 virus is an Omicron variant (such as Omicron BA.1.1.529).

13. A method according to any of claims 10 to 12, wherein said administration comprises intravenous administration.

14. A method according to any of claims 10 to 12, wherein said administration comprises nasal administration.

15. A method according to any of claims 10 to 12, wherein said administration comprises inhalation.

16. A method according to claim 15 wherein said inhalation is achieved using a nebulizer.

17. A lyophilized composition comprising a MASC protein of any of claims 1 to 5.

18. A composition comprising an antigen binding domain (ABD) that that binds to the trimeric Omicron BA.1.1.529 Spike protein, wherein said ABD binds to a first epitope of a first monomer and binds to a second epitope of a second monomer.

19. A composition comprising an ABD that binds to the trimeric Omicron BA.1.1.529 Spike protein, wherein said binding results in the RBD of said Spike protein being locked into the non-extended position.
